(19) United States
(12) Patent Application Publication
(10) Pub. No.: US 2005/0131442 A1
(43) Pub. Date: Jun. 16, 2005

(54) Insertion and retrieval system for inflatable devices

(76) Inventors: Daniel Yachia, Herzliya (IL); Eran Hirszowicz, Ramat Gan (IL)

Correspondence Address: NATH & ASSOCIATES, 1030 15th STREET, NW, 6TH FLOOR, WASHINGTON, DC, 20005, US

(21) Appl. No.: 10/507,612

(22) PCT Filed: Mar. 13, 2003
(86) PCT No.: PCT/IL03/00207

(30) Foreign Application Priority Data
Mar. 14, 2002 (IL) 148702

(51) Int. Cl. A61B 17/34

(52) U.S. Cl. 606/185; 606/192

(57) ABSTRACT

A system for deploying and retrieving implantable articles, such as balloons, typically for treating incontinence, from the bladder includes a urethral module, this deployed in the urethra and bladder with the coupling of a mandrel. The deployed urethral module, with the mandrel removed therefrom, provides a channel to the bladder for a magazine unit, that deploys the balloon, and a retriever, that retrieves the balloon, and is ultimately removed from the body with the balloon in a deflated or contracted state.

### FIELD OF THE INVENTION

The present invention is directed to devices for inserting and retrieving articles that are inflatable and deflatable into and from body cavities. In particular, these devices are directed to inserting and retrieving inflatable and deflatable articles that are configured for at least temporarily seating in the urinary r neck, whereupon this seating, block urine flow from the bladder.

### BACKGROUND OF THE INVENTION

There are many treatments for urinary incontinence in both males and females. One such treatment involves use of a balloon that temporarily seats in the bladder, blocking urine flow therefrom, as detailed in commonly owned U.S. Pat. No. 6,293,923 and U.S. patent application Ser. No. 09/594,641, both documents incorporated by reference in their entirety herein.

The balloons used in these systems are initially stored in deflated and unexpanded or contracted states. As a result of storage in this state, they may, upon long term storage, become creased and stressed, leading to a damaged balloon, whose inflation is problematic or partial, or not at all. These damaged balloons must be rejected, leading to increased costs with obtaining a new balloon.

### SUMMARY OF THE INVENTION

The present invention improves on the contemporary art by providing systems for insertion and removal of articles, typically balloons, that are of few pieces and easily operable. As such, these systems can be operated by multiple types of medical personnel, and are not limited to highly trained physicians. Moreover, the insertion devices that utilize balloons stored in inflated or expanded states. By storing the balloons in this manner, they are free of stresses and creases, assuring a high degree of structural integrity, reducing the chances of an unusable, and thus rejected balloon. Also, the operator can visually inspect the balloon in both the inflated and deflated states in the insertion device, eliminating the chances that a defective balloon will be deployed.

One embodiment of the present invention is directed to a system for deploying and retrieving implantable members, fillable articles (members), such as balloons, typically for treating incontinence, from the bladder. The system includes a urethral module, this deployed in the urethra and bladder with the coupling of a mandrel. The deployed urethral module, with the mandrel removed therefrom, provides a channel to the bladder, for a magazine unit, that deploys the balloon, and a retriever, that retrieves the balloon, and is ultimately removed from the body with the balloon in a deflated or contracted state.

Another embodiment of the invention is directed to a magazine unit. This magazine unit includes a body including a chamber configured for receiving an implantable device, typically a balloon, and the body including a passageway extending therethrough. There is a rod slidably received by the passageway in the body, this rod of a length for sliding beyond the body. The rod also includes a channel for fluid passage extending at least substantially therethrough. Also included is a channel member, typically a needle, in fluid communication with the channel, the needle configured for receipt of the implantable device.

Another embodiment of the invention is directed to a retrieving device, for retrieving the implanatable devices, typically a balloon. This retrieving device or retriever includes a body including a tubular segment extending therefrom, a capturing member engaged within the tubular segment defining a tip portion of the tubular segment; and a needle including a tip and a bore extending therethrough. The needle is coupled with a force generating member, for example, a spring, so as to be movable between a first position, where the needle tip has not moved beyond the tip portion, and a second position, where the needle tip has moved beyond the tip portion. This spring provides forces to the needle sufficient to pierce an implantable member or device, such as a balloon or other fillable article.

There is another embodiment directed to a method for deploying an implantable member or device, such as a balloon or other fillable device, in a body organ, such as the bladder. This method includes providing a magazine unit comprising: a body including a chamber configured for receiving an implantable member or device, the body including a passageway extending therethrough; a rod slidably received by the passageway in the body and of a length for sliding beyond the body, the rod including a channel for fluid passage extending at least substantially therethrough, and a channel member, for example, a needle, in fluid communication with the channel, the needle received in the implantable member. An implantable member in at least a partially expanded state is then provided onto the channel member; and the implantable member is contracted. Upon traveling to the bladder, the implantable member is then expanded (inflated), and once expanded it is released into the bladder, as the needle is retracted whereby a tip on the rod resists the implantable member upon retraction, ultimately releasing the implantable member into the bladder absent body forces.

Another embodiment is directed to a method for retrieving an implantable member (article, device, etc.), for example, a balloon or other fillable article from an organ, such as the bladder. This method includes accessing the bladder, for example, by creating a tubular channel thereto; capturing the implantable member, typically with a magnet as the capturing member; piercing the implantable member with a device in fluid communication with a fluid channel or conduit; and contracting (deflating) the implantable member by suctioning the contents of the member through the fluid channel or conduit.

### BRIEF DESCRIPTION OF THE DRAWINGS

Attention is now directed to the attached drawings, wherein like reference numeral or characters indicate corresponding or like components. In the drawings:

FIG. 1 is an exploded view of the system of the present invention;

FIG. 2A is a cross-sectional view of the urethral module in accordance with an embodiment of the present invention;

FIG. 2B is a perspective view of the urethral module of FIG. 2A;

FIG. 2C is a front view of the seal of the urethral module of FIGS. 2A and 2B;

FIG. 3A is a cross sectional view of the mandrel in a first position in accordance with an embodiment of the present invention;

FIG. 3B is a perspective view of the mandrel FIG. 3A;

FIG. 3C is a cross sectional view of the mandrel in a second position in accordance with an embodiment of the present invention;

FIG. 4A is a perspective view of the magazine unit in a first position in accordance with an embodiment of the present invention;

FIG. 4B is a cross-sectional view of the magazine unit in a second position in accordance with an embodiment of the present invention;

FIG. 4C is a perspective view of the magazine unit in a third position in accordance with an embodiment of the present invention;

FIG. 4D is a cross-sectional view of the magazine unit in a fourth position in accordance with an embodiment of the present invention;

FIG. 5A is a perspective view of the retriever in accordance with an embodiment of the present invention, in a first position;

FIG. 5B is a cross-sectional view of the retriever of FIG. 5A;

FIG. 5C is a perspective view of the retriever of FIG. 5A in a second position;

FIG. 5D is a cross-sectional view of the retriever of FIG. 5C; and

FIG. 6 is a cross-sectional view of the mandrel in the urethral module in accordance with the present invention, prior to insertion into the urinary tract;

FIGS. 7-9 are partial cross sectional and partial perspective views of instrumentation in accordance with the present invention in operation creating an instrument channel into the bladder;

FIGS. 10, 11, 12A, 13 and 14 are perspective views of the magazine unit and urethral module in accordance with the present invention, detailing processes associated with the deployment of an inflatable member or device;

FIG. 12B is a cross-sectional view of FIG. 12A taken along line 12B-12B;

FIGS. 15 and 16 are partial cross sectional and partial perspective views of instrumentation in accordance with the present invention in operation deploying the implantable member or device into the bladder;

FIG. 17 is a cross-sectional view of the deployed implantable member or device seated in the bladder neck;

FIGS. 18-20 are partial cross sectional and partial perspective views of instrumentation in accordance with the present invention in operation capturing implantable member or device in the bladder; and

FIGS. 21 and 22 are partial cross sectional and partial perspective views of instrumentation in accordance with the present invention in operation associated with the removal of the captured implantable member or device from the bladder.

### DETAILED DESCRIPTION OF THE INVENTION

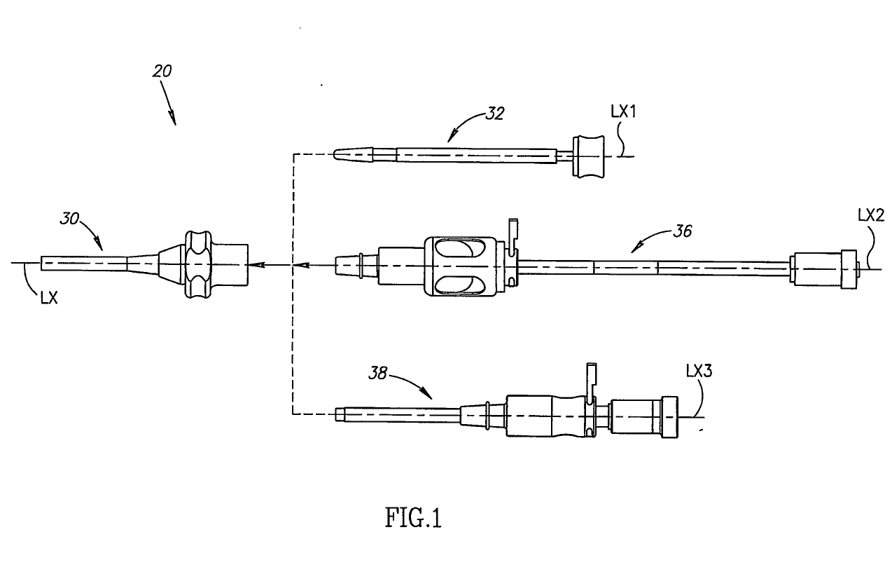

FIG. 1 shows the system 20 of the present invention with the relation of the components that form it. The system 20 includes a urethral module 30, having a longitudinal axis LX extending therethrough. The urethral module 30 receives a removable mandrel 32 (having a longitudinal axis LX1 extending therethrough), for example, in an aligned manner, where the longitudinal axes LX and LX1 are coaxial, that allows for placement of the urethral module 30 into the urinary tract, for example, here, the female urethra and bladder. With the mandrel 32, removed and the urethral module 30 in the urinary tract, either of the magazine unit 36 (having a longitudinal axis LX2 extending therethrough) or the retriever 38 (having a longitudinal axis LX3 extending therethrough) can be removably received by and placed through the urethral module 30, for example, in an aligned manner, where the longitudinal axes LX, and LX2 and LX3, respectively, are coaxial, to access the bladder for respectively inserting and inflating (collectively “deploying”), and retrieving an implantable device. The magazine unit 36 and retriever 38 can then be removed with the urethral module 30, typically as a unit, when the requisite insertions and retrievals of the implantable devices have been completed, as detailed further below.

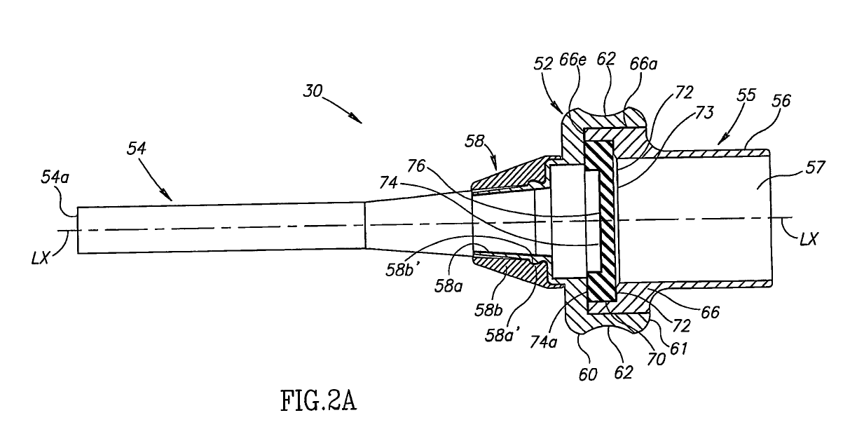

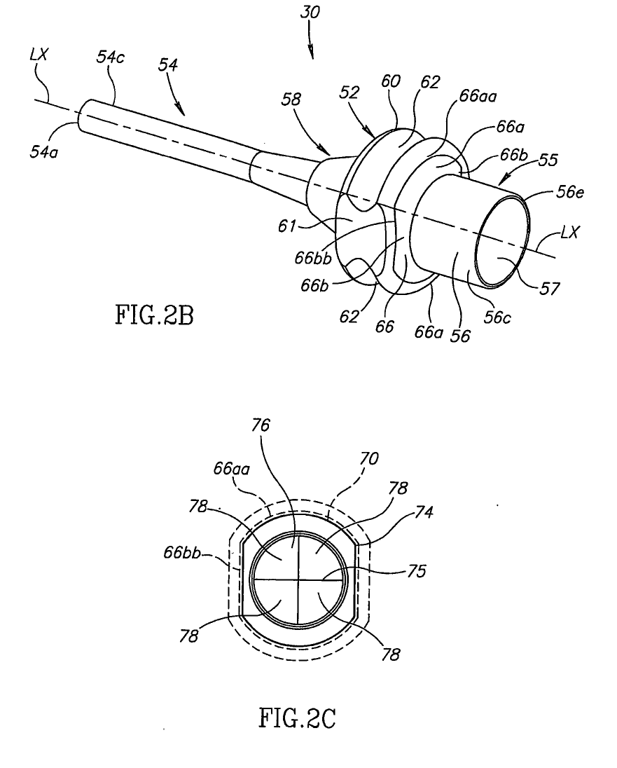

FIGS. 2A and 2B show the urethral module 30. This module 30 is typically formed of a body 52 and guide tube 54. The body 52 includes a receiver portion 55, with a cylindrical section 56 that defines a port 57. This cylindrical port 57 is configured to receive corresponding portions of the mandrel 32, magazine unit 36 and retriever 38, as detailed below.

The body 52 also includes a nose member 58, typically formed of an underlying nose 58a, and a nose cone 58b, for placement thereover. The nose 58a and nose cone 58b attach to each other by corresponding threads 58a′, 58b′ in a screw-like manner. This attachment holds the guide tube 54 in place by a clamping arrangement. The tapered shape of the nose cone 58b limits travel of the module 30 in the urethra.

The body 52 also includes a head 60 that includes a peripheral rim 61. This rim 61 includes indents 62, typically on both sides, for enhanced gripping by the fingers of the person performing the requisite procedure, with the urethral module 30.

The guide tube 54 includes an edge 54a, and is of a diameter large enough to support the corresponding shaft of the mandrel 32, and rods 132, 222 of the magazine unit 36 and the retriever 38, respectively, and the implantable device, typically in its deflated position. This diameter is of a size to minimize trauma to the urethra upon its insertion.

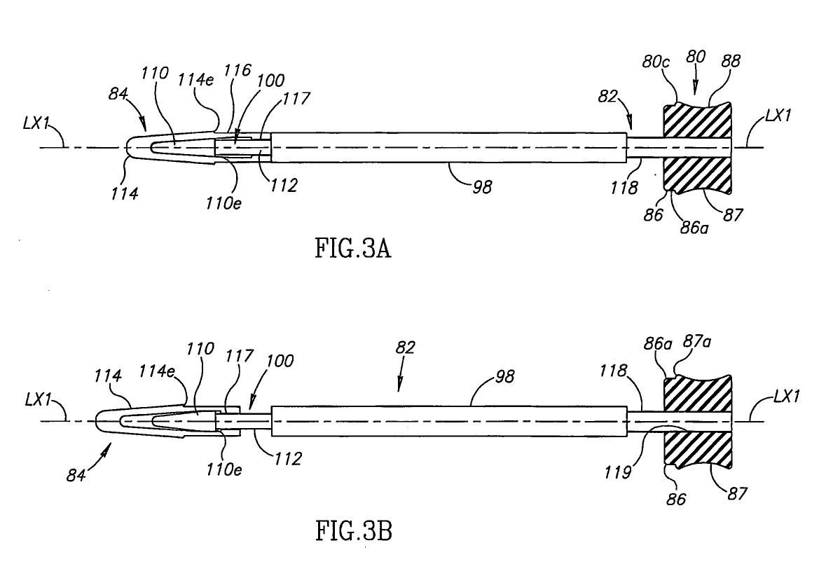

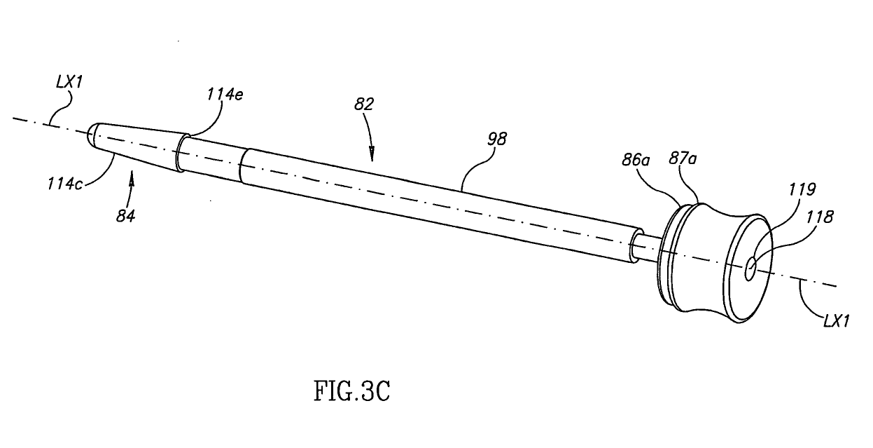

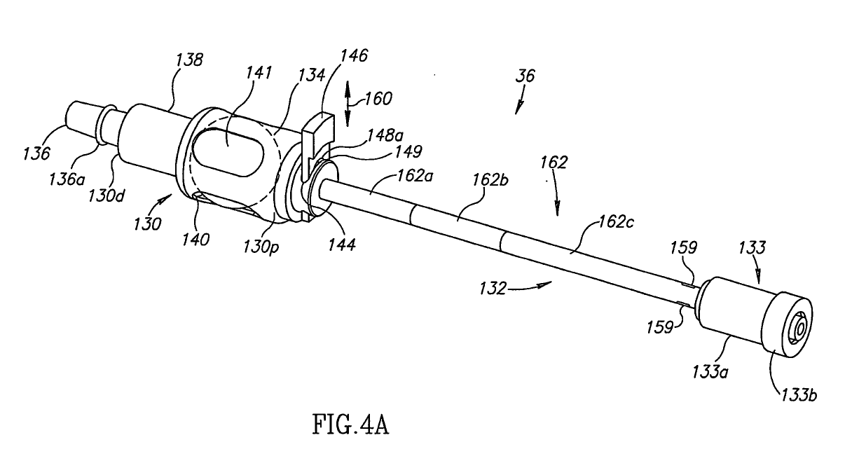

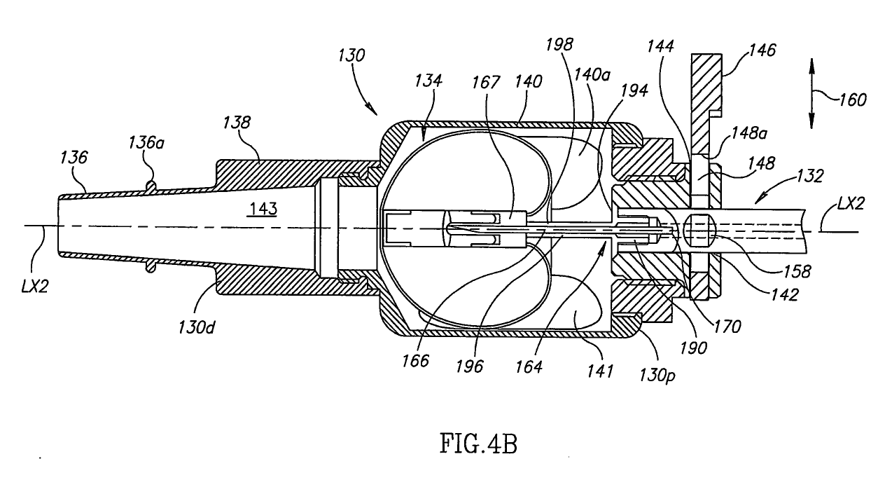

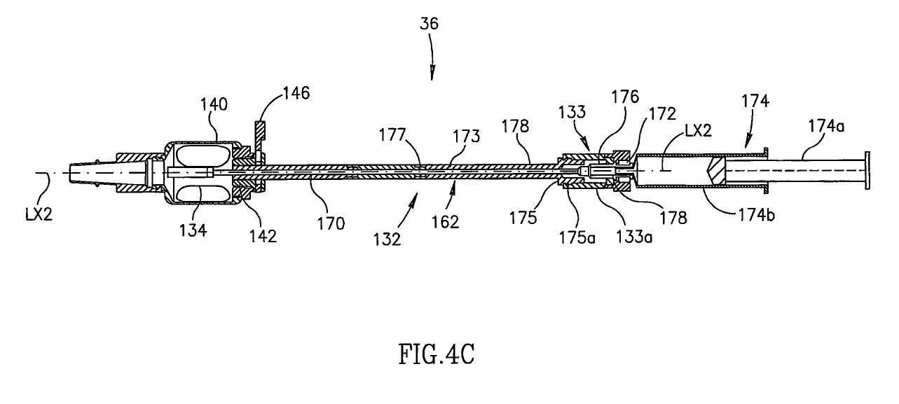

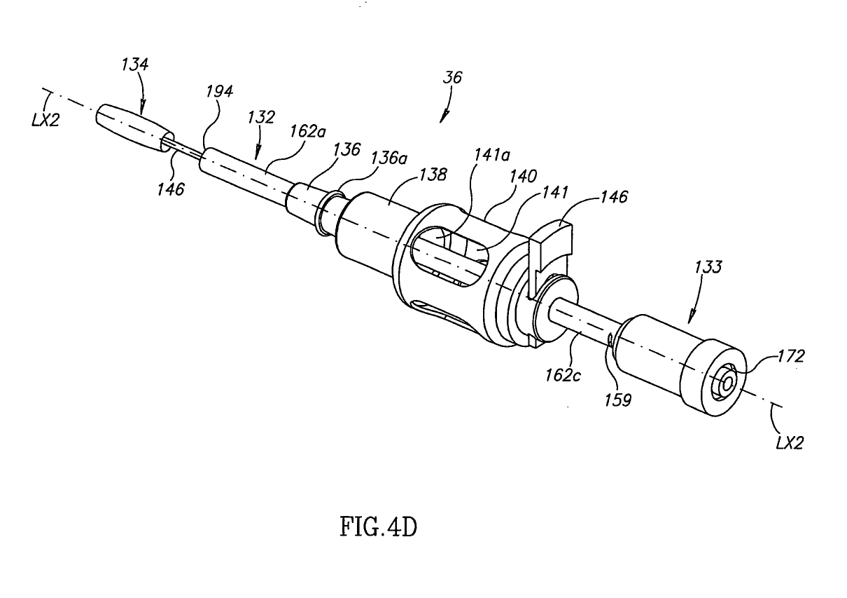

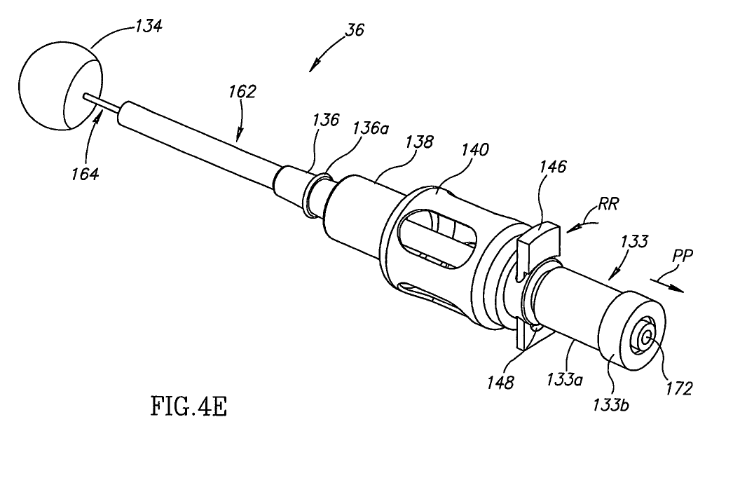

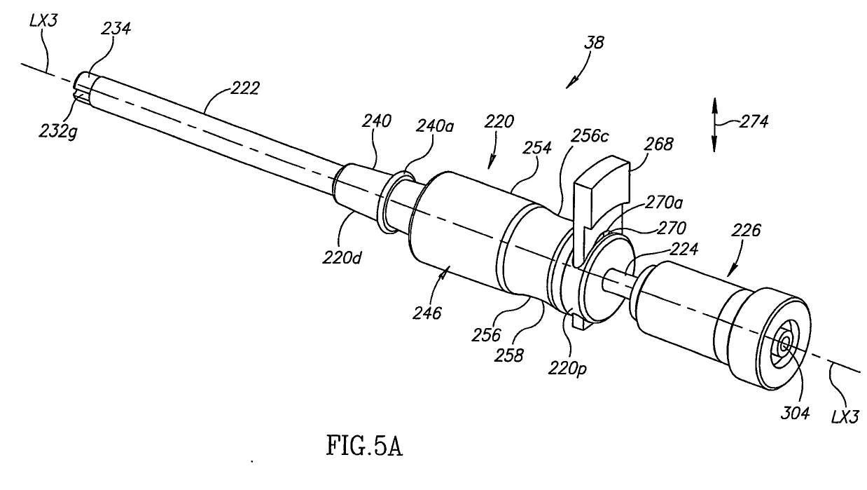

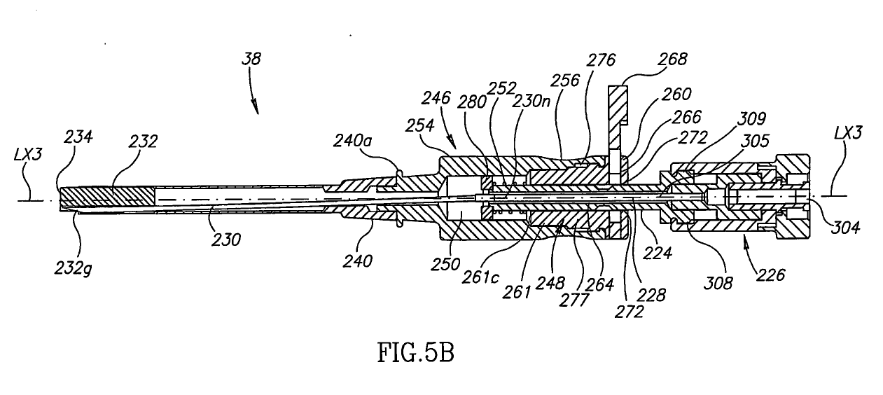

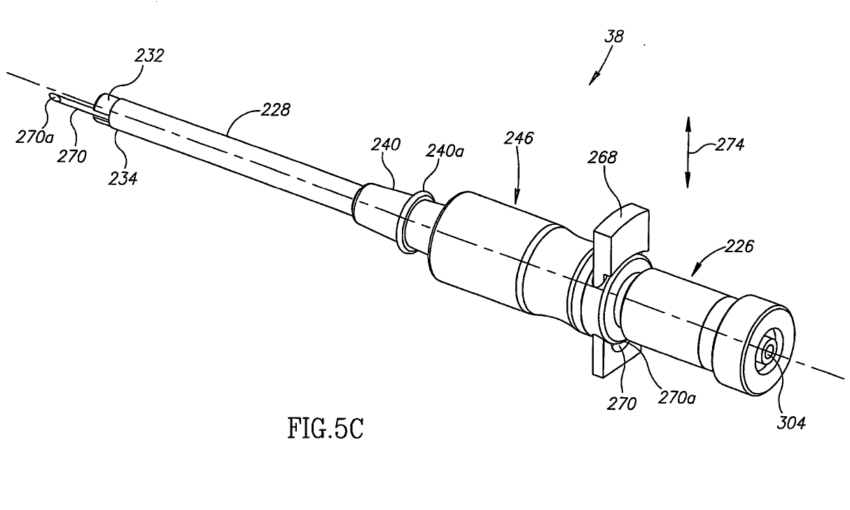

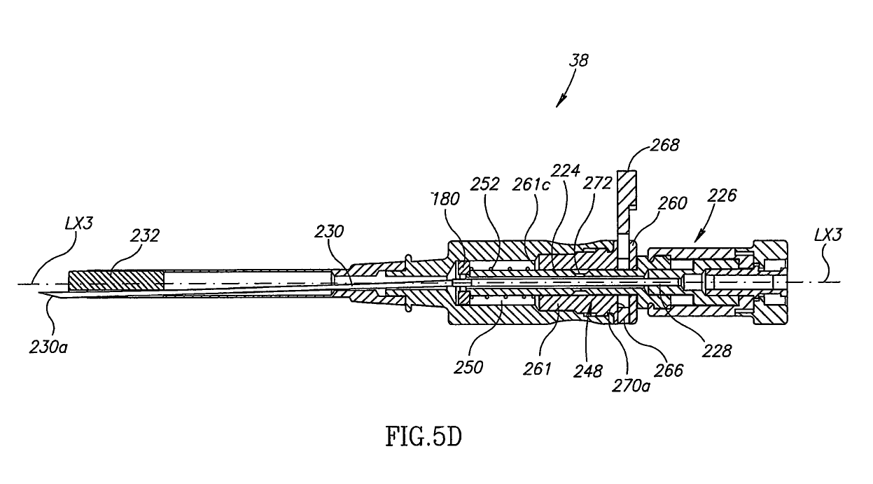

The receiver portion 55 is configured to connect within the body 52 at the rim 61, in a frictionally tight manner. Similarly, the cylinder section 56 defining the port 57 is of an internal diameter to receive the head 80 of the mandrel 32, at its preliminary diameter portion 86a (FIGS. 3A-3C), and the neck portion 134 of the magazine unit 36 (as shown in FIGS. 4a-4e) and the distal segment 254 (as shown in FIGS. 5a-5d) of the retriever 38 in a stable manner in a frictional engagement tight enough to seal against urine leakage. This cylindrical section 56 also includes an outer surface 56c and an edge 56e.

The receiver portion 55 also includes a collar 66, with rounded 66a and straight 66b sides. A ledge 70 is formed by inner walls 66aa and 66bb of the rounded 66a and straight 66b sides, and the walls 72 surrounding a circular bore 73 (as shown in FIG. 2A). The circular bore 73 is at the junction of the collar 66 and cylinder section 56, as is of a diameter, for example, smaller than the inner diameter of the cylinder section 56. This wall 72 defines the diameter of the circular bore 73, and serves as a stop surface for the magazine unit 36 and retriever 38, limiting their respective travel through the urethral module 30 (as detailed below).

The body 52, and its receiver portion 55 are formed, for example of medical grade polymers. These medical grade polymers may be for example, polyethylene, polypropylene, TEFLON® or the like. The guide tube 54 is typically formed of a thin polymer, for example, TEFLON® or silicon.

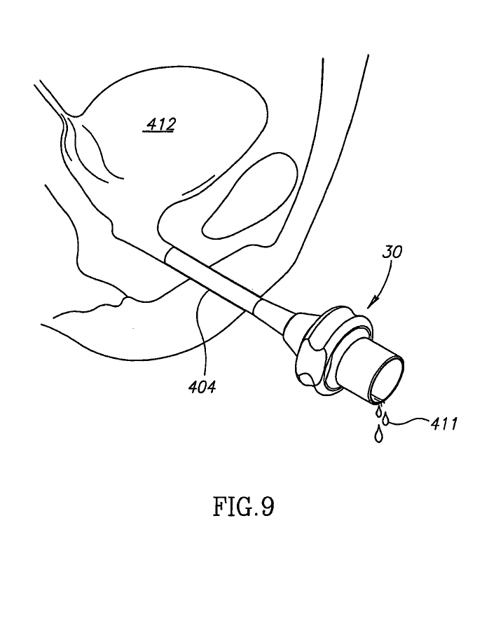

The ledge 70, receives a seal 74, that is dimensioned, for example, to have a perimeter just slightly smaller that the corresponding perimeter of the inner walls 66aa, 66bb of the collar 66. This dimensioning allows the seal 74 to seat in the collar 66, in a frictional engagement (although additional securement with adhesives is also permissible). The seal 74, shown in detail in FIG. 2C, protects against massive leakage of urine, but allows for passage of drops 411 (FIG. 9) of urine from the urethral module 30, when it is properly deployed (as detailed below and shown in FIG. 9).

The seal 74 has, for example, cuts 75 that extend through it in a crosshair pattern. These cuts 75 are typically in a circular area 76 on one side of the seal 74, and divide the seal 74 into panels 78. This configuration accommodates the shape of the noses of the magazine unit 36 and the retriever 38, by allowing for penetration of the magazine unit 36 and retriever 38 therethrough, while capturing these instruments to prevent them from moving backwards in the urethral module 30. The edge 74a of the seal 74 is typically flush with the edge 66e of the collar 66. The seal 74 is typically made of a medical grade silicone, or other elastomeric material.

The mandrel 32 is shown in FIGS. 3A-3C. FIGS. 3A and 3C show the mandrel 32 in a first position, associated with insertion (in cooperation with the urethral module 30 as detailed below), while FIG. 3B shows the mandrel 32 in a second position, associated with its removal from the urethral module 30 (detailed below).

The mandrel 32 includes a head 80, and a shaft 82, with the shaft 82 capped by a flexible tip 84. The head 80 includes a first section 86 and a second section 87. The first section 86 is of a preliminary diameter portion 86a, that is typically of a smaller diameter than the second section 87. This preliminary diameter portion 86a of the first section 86 is slightly smaller than that of the inner diameter of the cylindrical section 56 of the receiver portion 55. This configuration allows for the shaft 82 of the mandrel 32 to be aligned in the guide tube 54 of the urethral module 30.

At least a portion, typically the distal edge 87a, of the second section 87 is of larger diameter then the inner diameter of the cylindrical section 56 of the receiver portion 55. This dimensioning allows the proximal edge 56e (FIG. 2B) of the cylindrical section 56, to serve as a stop surface for the head 80, limiting travel of the shaft 82 in the urethral module 30 (detailed below). For example, the diameter of the second section 87, at the distal edge 87a can be equal to the outer diameter of the of the cylinder section 56, of the receiver portion 55, so that the outer surfaces 56c, 80c, of the cylindrical section 56 and head 80 are flush.

The head 80, and in particular its outer surface 80c, can also be contoured. This contouring is typically in the form of an indented rounded ring 88. This indented rounded ring 88 allows for enhanced gripping by the fingers of the person performing the procedure.

The shaft 82 is typically of a stiffness necessary to penetrate and move within the urethra and bladder (as detailed below). The shaft 82 is of a diameter less than that of the guide tube 54 of the urethral module 30, for sliding therethrough. The shaft 82 has a main portion 98 of a hard material, with a central pole 100 extending therefrom. The flexible tip 84 is of an inner dimensioning to be frictionally fit over this pole 100, this friction fit of a tolerance that allows movement of the tip 84 to slide along the pole 100 in directions parallel to it (to the longitudinal axis LX1).

The pole 100 is formed of a tapered portion 110 on a rod 112. The tapered portion 110 extends beyond the diameter of the rod 112, forming an edge 110e. The flexible tip 84 includes a corresponding tapered end 114 joined to a cylindrical section 116. The cylindrical section 116 includes an inner lip 117, for contacting the edge 110e when the flexible tip 84 moves away from the main portion 98, such that the flexible tip 84 remains on the pole 100, and does not fall off of the pole 100 during removal of the mandrel 32 from the guide tube 54 (of the urethral module 30), as shown in FIG. 3C (illustrative of a second position for the mandrel 32).

Similar to the tapered portion 110, the tapered end 114 extends to a diameter beyond that of the cylindrical section 116, forming an edge surface 114e at the junction of the tapered end 114 and the cylindrical section 116 (FIG. 3A). This diameter of the tapered end 114 is typically equal to that of the diameter of the guide tube 54. By providing this flexible tip 84 for moving on the pole 100 of the shaft 82, it reduces trauma to the urethra by moving to a smaller diameter, upon its removal from the guide tube 54 of the urethral module 30, as shown in FIG. 3C. This edge surface 114e is of a perimeter thickness, sufficient for abutting the edge 54a of the guide tube 54. The outer diameters of the tapered end 114 and guide tube 54 are typically equal, such that the outer surfaces 114c, 54c (FIG. 2B) of respective tapered end 114 and guide tube 54 are flush. This flushness keeps the edge 54a of the guide tube 54, that can be sharp, from contacting urethral tissue, avoiding damage and trauma to it.

Accordingly, when mandrel 32 is removed from the urethral module 30, (FIG. 3B) the abutment of the edge surfaces 114e and 54a causes the flexible tip 84 to move apart from the main portion 98. Upon this movement, a portion of this flexible tip 84 has a smaller diameter than the guide tube 54, allowing the remainder of the flexible tip 84 to be pulled through the guide tube 54 upon removal.

The shaft 82, and in particular, the main portion 98 connects to the head 80 by a threaded rod portion 118. This threaded rod portion 118 is typically integral with the main portion 98 and is received by a correspondingly threaded section 119 in the head 80, and as such, allows the shaft to be adjustable, whereby the flexible tip 84 can be adjusted to accommodate for individual guide tubes 54, and in particular their edges 54a, such that the outer surfaces 114c and 54c are flush (as detailed further herein).

The pole 100, and shaft main portion 98 can be separate or integral members. In the case that they are separate members, they can attach to each other by corresponding screw threads or the like. All components of the mandrel 32, except the flexible tip, are typically made of the medical grade polymers as detailed above. The flexible tip 84 is typically made of an elastomeric material, such as medical grade silicone, and is of a hardness less than that for the pole 100 and shaft main portion 98.

FIGS. 4A-4E detail the magazine unit 36. FIGS. 4A and 4B show the magazine unit in a first position where the implantable device, member, article, or the like, is stored. FIG. 4C shows the magazine unit 36 in a second position, where the implantable device has been deflated or contracted, so as to be ready for deployment. FIG. 4D shows the magazine unit in a third position, where the implanatable device is in a proper position for deployment, prior to its expansion or inflation and FIG. 4E shows the magazine unit 36 in a fourth position, where the implantable device has been expanded or inflated prior to its release into the body.

The magazine unit 36 includes a main body 130 that receives a rod 132, slideable within this main body 130, the rod connecting to a head 133. This slideable arrangement of the rod 132 in the main body 130, allows for inserting of an implantable member (device), for example, a balloon 134, or other member or device into the urinary tract, that can block urine flow from the bladder.

The balloon 134 is typically a balloon or other fillable article or member, that can be inflated or expanded and deflated or contracted. The balloon 134 can be for example, a balloon or other article or device, as detailed in U.S. Pat. No. 6,293,923, the disclosure of which is incorporated by reference in its entirety herein. This balloon is typically fillable with fluids, such as gases, liquids, gels, etc.

Other balloons and other fillable articles, suitable for use herein are typically inflated or filled with fluids, including gases, liquids, gels, etc. These balloons, for example, typically include magnetic or magnetizable materials therein. These balloons are such that when inflated or expanded in the bladder, these balloons can be positioned to seat in the bladder neck, so as to block the flow of urine (as detailed below). Positioning can be either by sinking (should the balloon have a specific density greater than that of urine), so as to fall in the bladder neck, or held in place there by a magnet in an undergarment or the like (should the balloon be of specific density less than that of urine).

The main body 130 includes a distal end 130d and a proximal end 130p. The distal end 130d includes a nose portion 136 and a neck portion 138 (FIG. 4A).

The nose portion 136 includes a collar 136a, typically correspondingly shaped (here for example, outwardly tapered) with respect to the nose 58a in the urethral module 30. This nose portion 136 is received in the nose 58a, with the collar 136a serving to catch the seal 74, to prevent backward movement of the magazine unit 36 when in the urethral module 30.

Similarly, the neck portion 138 is typically cylindrical in shape and of a diameter slightly less than that of the inner diameter of the cylindrical section 56 of the receiver portion 55, so as to allow for sliding within the cylindrical section 56. This dimensioning is such that engagement of the neck portion 138 by the cylindrical section 56 stabilizes the magazine unit and seals the components of the magazine unit 36 against leakage of urine (once its nose portion 136 has moved past the seal 74 in the urethral module 30).

A magazine section 140 is formed in the main body 130, intermediate the distal 130d and proximal 130p ends. This magazine section 140 is proximate to the neck portion 138, and has a cavity 140a dimensioned to accommodate, the balloon 134 in an inflated or expanded state in order to allow its storage and subsequent deployment, as detailed herein (FIG. 4B). The magazine section 140 includes openings 141, typically covered with a transparent, clear or other see-through sheath 141a, that allows for viewing (and in some cases a visual inspection) of the balloon 134.

The proximal end 130p of the main body 130 includes a bore 142, continuous with the magazine unit cavity 140a and the cavity 143 formed by the interiors of the nose 136 and neck 138 portions, through which the rod 132 slides. This proximal end 130p also includes a slot 144, through which a tongue 146 slideably extends through. The tongue 146 includes an opening 148, with edges 148a, for engaging distal 158 and proximal 159 notches in the rod 132. These notches 158, 159 are typically paired or in series, and disposed approximately 180 degrees from each other on the rod 132. There can also be single notches on the rod 132 at these positions.

The tongue 146 can be slid within the slot 144 (in the direction of double headed arrow 160), such that its edges 148a engage one of the respective distal 158 and proximal 159 notches, locking the rod 132 in position. Sliding the tongue 146 in the slot 144, such that the engagement is broken (also as per arrow 160), allows the rod 132 to slide freely in the body 130 of the magazine unit 36. The notches 158, 159 are at positions where the rod 132 can be locked, whereby operations of the magazine unit 36 (detailed below) can be performed with safety.

The rod 132 has a shaft 162 and a tip 164 at its distal end. The tip 164 (FIG. 4B) receives a channel member, such as a needle 166 (with a bore extending therethrough, allowing for fluid transport therethrough for inflation/deflation of the balloon 134), this needle 166 received by the balloon 134, and in particular in a valve in a magnetic or magnetically attractive portion 167, typically defining the central core of the balloon 134.

A tube 170 (FIG. 4C), attached to the head 133, extends from the head 133, through the shaft 162 to a point proximate the tip 164, this tube 170 receiving the needle 166, typically by a frictional engagement (with this additional securement with adhesives, and the like also permissible). The tube 170 is of an inner diameter slightly larger then the outer diameter of the needle 166, so as to receive the needle 166 in a frictional engagement.

This arrangement of the needle 166, tube 170, and port 172, at the proximal end of the head 133, defines a channel or conduit 173, such that fluid, including air, liquid, gel or the like, can be suctioned from or placed into the balloon 134, or the like (depending on the particular process). The port 172 is configured for typically receiving a syringe 174 (typically formed of a plunger 174a received in a body 174b), or any other suction or inflation mechanism(s), detailed additionally below.

The tube 170 can be for example, a metal tube, with a polymeric port 172 (to provide friction for receiving the syringe 174). Alternately, the needle 166 and tube 170 can be a single or integral member, for example, a single needle having a bore extending therethrough.

The head 133 is of a diameter larger than the bore 142, so as to limit travel of the rod 132, in the distal direction (FIGS. 4C-4D) The head 133 joins to the shaft 162, in particular a collar 175 on the shaft 162, by threads 133a, that engage corresponding threads 175a on the collar 175 in a screw-like manner. The head 133 can be separated from the collar 175 (typically by rotating it in the direction indicated by the arrow RR) (FIG. 4E). Upon this separation, the head 133 can be pulled proximally (in the direction of arrow PP). As the head 133 is attached to the tube 170, that receives the needle 166 in a frictional engagement, the needle 166, upon this proximal pulling, moves proximally into the tip 164. Continued proximal movement results in the disengagement (release) of the implantable member (device), here for example, the balloon 134, from the needle 166 without any forces on the body of the patient, who is receiving the implantable member (device).

The head 133 can be formed of additional or multiple pieces. These pieces can be joined together by screw type mechanisms or the like.

The tube 170, typically in the shaft 162, also includes a protruding ring or alternately, rings 177 (FIG. 4C) of a diameter larger than that of the tube opening 178 in the shaft 162 or collar 175. The larger diameter ring(s) 177, limit travel of the tube 170 and needle 166, such that they are not pulled out of the rod 132, when the head 133 is pulled proximately (typically during retraction of the needle 166 upon release of the implantable member, here for example, the balloon 134).

The shaft 162 includes the notches 158, 159, that receive the portions of the tongue 146, when locking (as detailed above), typically for safety, is desired. The distal notches 158 are, for example, positioned such that locking is at a position where there is sufficient space for the balloon 134 in the magazine section 140, such that it can be stored in an expanded state, so as to minimize potential damage from storage. The proximal notches 159 (FIG. 4D) are, for example, positioned such that the shaft 162 can extend a sufficient distance through the distal end 130d and into the urethra and bladder, so as to properly insert and inflate (deploy) the balloon 134 in the bladder (FIG. 4E).

The shaft 162 can be, for example, formed in pieces, here three pieces 162a-162c (FIGS. 4A-4C). These pieces 162a-162c can include series of corresponding threads, each joining together in accordance with a screw mechanism.

Turning specifically to FIG. 4B, the tip 164 includes a tail 190, typically threaded, so as to be received in the shaft 162 (piece 162a that is correspondingly threaded) in a screw-like engagement, a platform 194, and a neck 196, extending from the platform 194. This tip 164 is typically a one-piece integral member formed of the medical grade polymers detailed above. A bore 198 extends through this tip 164, so as form a passage and support for the needle 166, that connects to the tube 170.

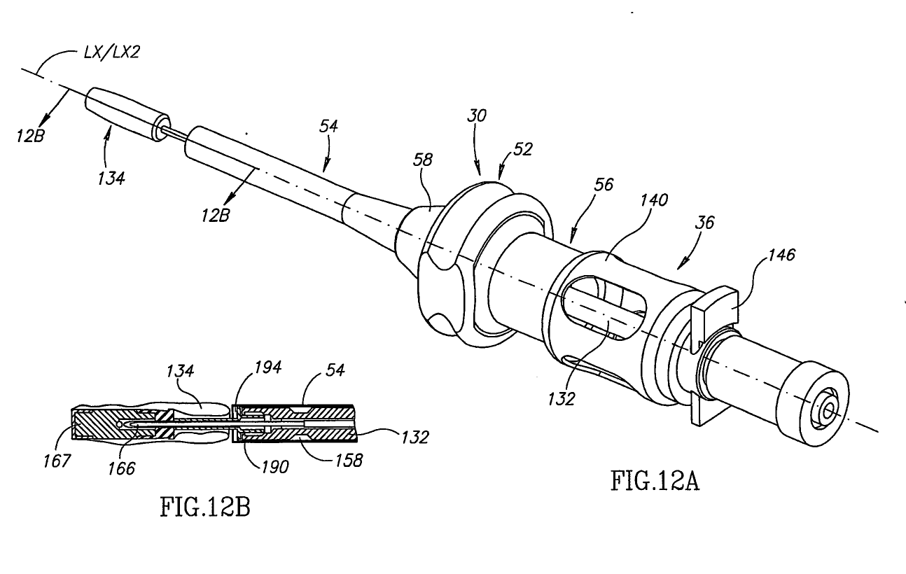

The platform 194 is of a diameter slightly greater than the bore 142 in the main body 130, so as to keep the rod 132, from sliding out of the body 130. However, it is much less then the diameter of the guide tube 54. This dimensioning provides room for balloon 134 portions that may move proximally and into the space between the platform 194 and guide tube 54 when the balloon 134 is moved distally for its insertion, so as not to be pinched and damaged upon this distal movement in the guide tube 54 of the urethral module 30 (also shown in FIGS. 11, 12A and 12B).

The body 130 and rod 132, and all components thereof, except where specifically mentioned, are typically formed from several pieces (as detailed above). These pieces, are made of medical grade polymers, as detailed above. The needle 166 and tube 170 and ring 177 are, for example, made of metal, such as surgical grade metals.

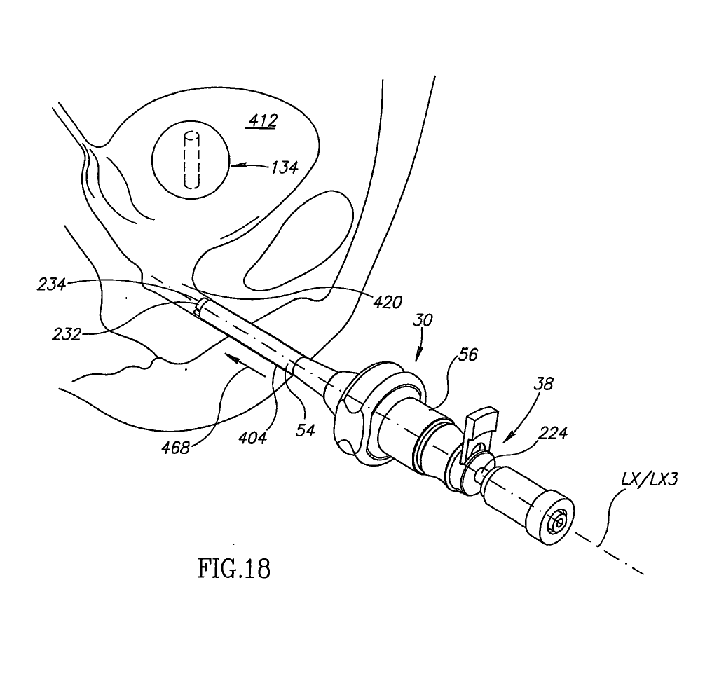

FIGS. 5A-5D show the retriever or retriever member 38 in detail. FIGS. 5A and 5B show the retriever 38 in a first position, suitable for capture of the implanatable member article in the body (here, for example, the balloon 134 in the bladder 412 as shown in FIGS. 18-22 and described below). FIGS. 5C and 5D show the retriever 38 in a second position, where it punctures the implantable article (e.g., the balloon 134) for its deflation or contraction, in order that the implantable article be removed from the body with minimal trauma thereto.

The retriever 38 includes a body 220, with distal 220d and proximal 220p ends, and a support tube 222 extending distally therefrom. The body 220 receives a rod 224, that slides therein, the rod 224 having a head 226 at its proximal end (FIGS. 5A-5B). The head 226 is similar in construction to the head 133 detailed above. A tube 228 extends from the head 226 and into the rod 224, where it receives a needle 230 (having a bore extending therethrough). The support tube 222 typically includes a capturing member, here for example a magnet or magnet attracting member 232 (hereinafter, “magnet”), defining its tip 234 (and also the tip of the retriever 38). The magnet 232 typically includes a groove 232g, for receiving the needle 228 and providing a track for it, when it is extended from the tip 234.

The distal end 220d of the body 220 includes a nose portion 240. The nose portion 240 includes a collar 240a, similar to the collar 136a of the magazine unit 36 detailed above, that prevents backward movement of the retriever 38 in the urethral module 30.

The proximal end 220p of the body 220 includes a main portion 246 that typically receives a backing member 248, for example in a screw-like engagement. The main portion 246 is typically open or hollow (defining a chamber 250) for accommodating a nose segment 261 of the backing member 248, a force generating member, typically a spring 252, and a plate 280 of the rod 224, all detailed further below. The main portion 246 includes a distal segment 254, and a proximal segment 256.

The distal segment 254 is similar to the neck portion 138 of the magazine unit 36, as it is typically cylindrical in shape and of a diameter slightly less than that of the cylindrical portion 56 of the receiver portion 55, so as to allow for sliding within the cylindrical portion 56. This dimensioning is such that engagement of the cylindrical portion 56 and the distal segment 254 will also serve to stabilize the retriever 38 upon this engagement, and seals the retriever 38 against leakage of urine (once its nose portion 240 has moved past the seal 74 in the urethral module 30).

The proximal segment 256, in particular its outer surface 256c, can also be contoured, similar to the contour ring 88 of the head 80 of the mandrel 32 (detailed above). This contouring is typically in the form of an indented rounded ring 258. This indented rounded ring 258 allows for enhanced gripping by the fingers of the person performing the procedure.

The backing member 248 includes a disc segment 260, typically integral with a nose segment 261, with a continuous bore 264 therethrough. This bore 264 is of a diameter slightly larger than that of the rod 224, to allow the rod 224 to slide therein. The disc segment 260 includes a slot 266, through which a tongue 268 slideably extends through. The tongue 268 includes an opening 270 with edges 270a for engaging notches 272 (at least one notch but typically two, arranged in a manner similar to notches 158, 159 in magazine unit 36 as detailed above) for locking and unlocking the rod 224 when the tongue 268 is slid in the directions of the double headed arrow 274. The tongue 268, slot 260, notches 272 are similar to those for the magazine unit 36 detailed above.

The nose segment 261 typically includes threads 276 at one end, for connecting with corresponding threads 277 on the main portion 246 of the body 220, to hold the backing member 248 in place, allowing the rod 224 to slide proximally and distally, moving the needle 230 (into and out of the support tube 222), when the spring 252 is contracted (compressed) and relaxed (by expansion) as detailed below. The other end of the nose segment 261 is of a flat surface 261c, and is of a diameter larger than the rod 224, and serves as one confinement for the spring 252.

The rod 224 extends distally beyond the backing member 248, where it terminates in a plate 280. The spring 252 extends along this portion of the rod 224 to this plate 280, that is of a larger diameter then the rod 224. The plate 280 serves as the other confinement for the spring 252.

The tube 228 connects to the needle 230 proximate this plate 280, with the needle origin 230n concentric and coaxial with the rod 224 and tube 228. The needle 230 is received by the tube 228, typically in a friction fit, and can also be joined to the tube 228 by adhesives, other fastening mechanisms or combinations thereof. The needle 230, tube 228 and port 304 (detailed below) define a channel or conduit 305, for fluid transport through the retriever 38. The needle 230 extends distally from this plate 280 and through the support tube 222, typically at an angle with respect to the longitudinal axis LX3, so as to be received by the groove 232g in the tip 234. For example, the needle 230, in this orientation, will pierce (puncture the outer shell and penetrate into the interior chamber) the balloon 134 at a location off center, to the side of the magnet central core 167. Alternately, the needle 230 and tube 228 can be a single member, for example, a single needle with a bore extending therethrough.

The needle 230 is initially in the support tube 222, when the spring 252 is in the compressed position (FIGS. 5A-5B). This compression is retained, as the tongue 268 engages a notch 272, creating a locking for safety. When unlocking from this locked position is desired, the tongue 268 is slid in the down direction of the arrow 274, breaking the contact, allowing the spring 252 to expand, resulting in the needle 230, in particular the needle tip 230a, moving out of the support tube, beyond the tip 234 (FIGS. 5C-5D). The spring 252 is such it provides a force for propelling the needle 230 to easily penetrate the balloon 134. The needle 230 has a hollow interior of a bore diameter such that the balloon filling material can rapidly flow through the needle 230, without regard to its viscosity, allowing the balloon 134 to drain and deflate (contract) rapidly.

The rod 224 terminates in the head 226. The tube 228 within the rod 224 extends to a port 304 in the head 226. This tube 228 is in communication with the needle 230, such that fluid, including air, liquid, gel, or the like can be suctioned from or placed into the balloon 134, or the like, once it has been captured, as detailed below. The port 304 is configured for typically receiving a syringe (similar to the syringe 174 detailed above), or any other suction or inflation mechanism(s), detailed additionally below.

The head 226 is of a diameter larger than the bore 264, so as to limit travel of the rod 224, in the distal direction. The head 226 may join to the rod 224, by correspondingly positioned threads 308, 309, that create a screw-like engagement. The head 226 is similar in construction to the head 133 detailed above.

The body 220, rod 224, head 226, and all components thereof, except where indicated, can be formed of any number of pieces, with some pieces being combined to be integral pieces. These pieces are made, for example, of the medical grade polymers detailed above. The tube 228 and needle 230 are for example, integral pieces and typically made of surgical grade metals or the like. The magnet or magnetically attractive member 232 is for example made of magnetic metals or magnetically attractive metals. The port 304 is typically of a polymeric material, to provide friction for receiving the syringes or the like.

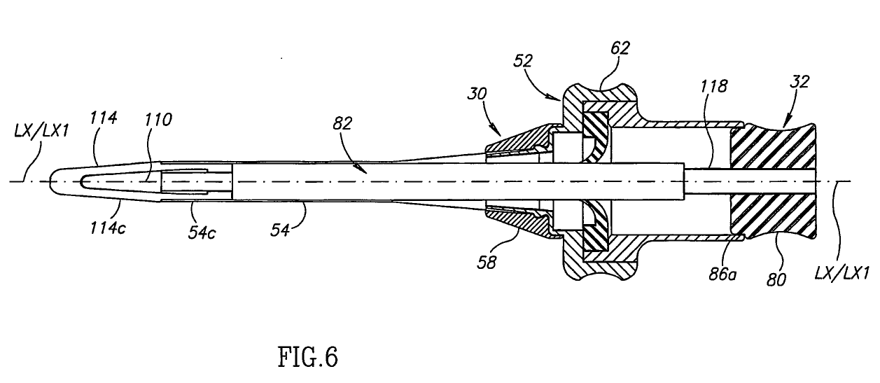

FIGS. 6-20 detail the system 20 in an example operation in the urinary tract of a human female. In these figures reference is made to the urethral module 30, mandrel 32, magazine unit 36 and retriever 38, and components thereof, as detailed above and shown in the previous drawing figures.

As shown in FIG. 6, the mandrel 32 is placed within the urethral module 30. There may be an adjustment of the tip 114 with respect to the guide tube 54 of the urethral module 30, such that they abut in alignment and their outer surfaces 114c and 54c are flush. This is performed by rotating the head 80 on the threaded rod portion 118 of the shaft 82.

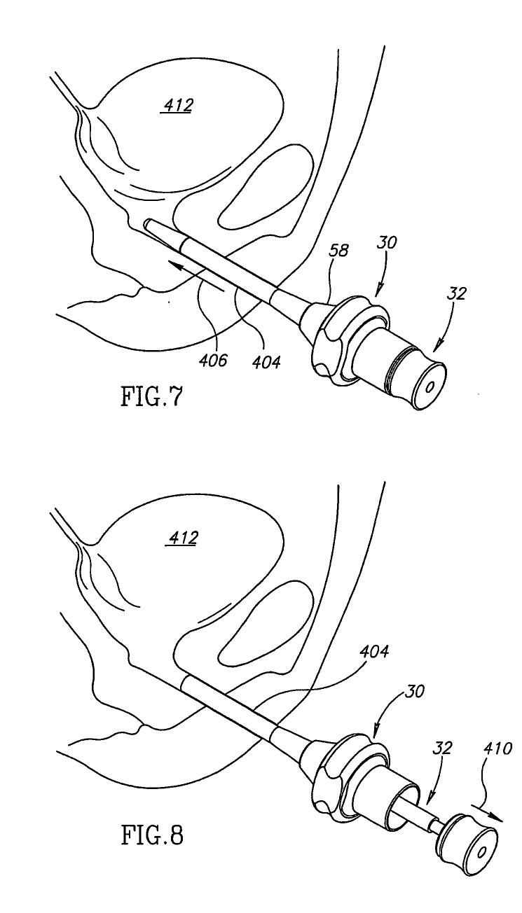

The now combined devices 30, 32 are now gripped by the person inserting them, and inserted into the female urethra 404, in the direction of the arrow 406 (FIG. 7). Insertion continues until movement becomes difficult, typically from resistance by the widening of the nose 58 of the urethral module 30, as shown in FIG. 7.

In FIG. 8, the mandrel 32 is then removed, by moving it in the direction of arrow 410, while the urethral module 30 is held in place. In this moving, the tip 84 (in particular the tapered portion 114) of the mandrel 32 contacts the edge 54a of the guide tube 54 and moves distally, so as to decrease its diameter to lessen trauma, as it subsequently pulled into and through the guide tube 54 of the urethral module 30.

In FIG. 9, the urethral module 30 remains in the urethra 404, and its proper placement can be confirmed as drops of urine 411 can be detected by leaking through the seal 74. The now deployed urethral module 30 provides a channel therethrough, for instrumentation to access the bladder 412.

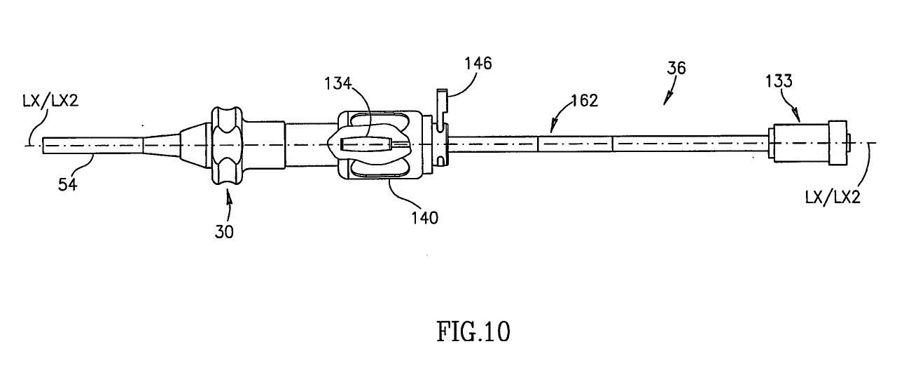

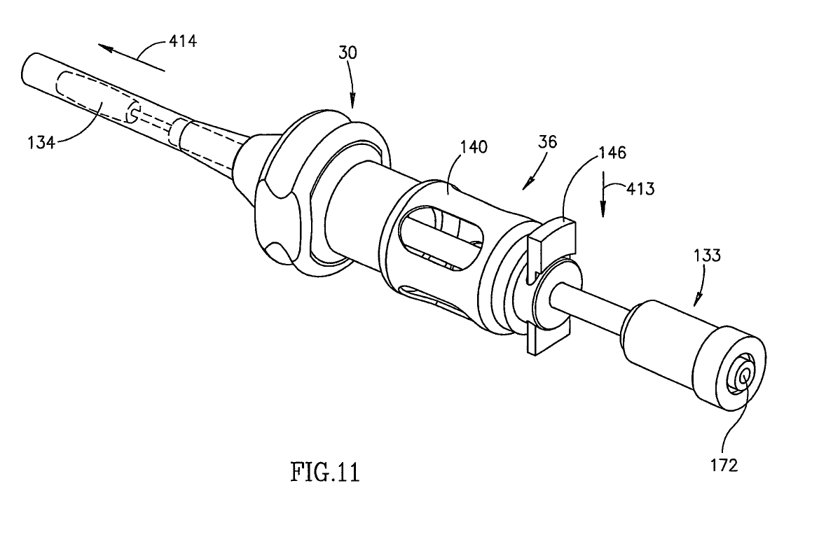

In FIG. 10, the magazine unit 36 is then made ready for placement into the now deployed urethral module 30. This process begins as a syringe is then connected to the port 172 of the magazine unit 36 and the fluid, typically air, is suctioned from the balloon 134, such that it contracts (deflates) (FIG. 4C). In FIG. 11, the previously locked rod 132 is unlocked as the tongue 146 is moved down, in the direction of the arrow 413, out of engagement with the distal notch 158. The rod 132 is now released and this now contracted balloon 134 is pushed distally for its deployment, by pushing the rod 132 distally (in the direction of the arrow 414).

Pushing (distally) on the rod 132 continues, until the rod 132 cannot be further pushed or the balloon 134 is outside (beyond) he guide tube 54, as shown in FIG. 12A. During this proximal movement, the balloon 134 may include portions that sagged over the needle 166 and tip 164, with this sagging accommodated by the space between the guide tube 54 and the tip 164, as also shown in FIG. 12B. Here, the balloon 134 has entered the bladder.

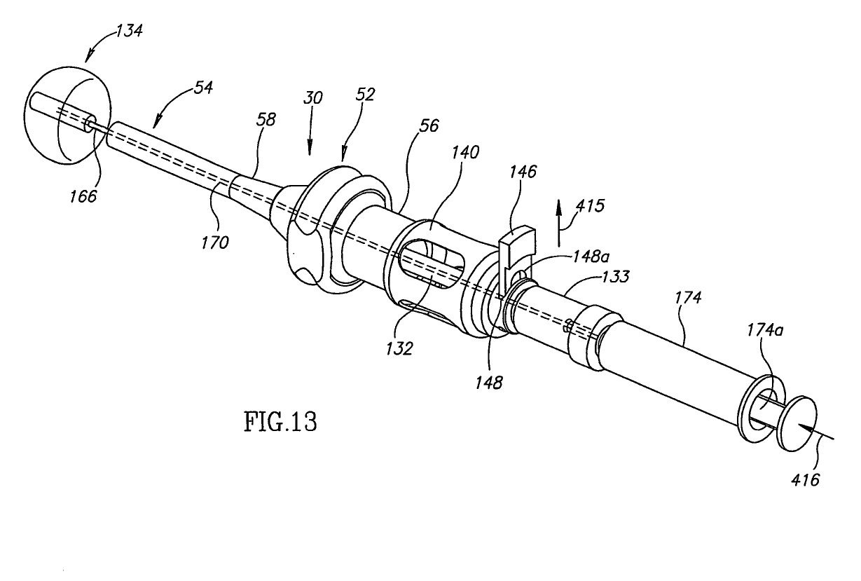

In FIG. 13, the balloon 134 is now in position for inflation (expansion). The rod 132 is now locked in a safety position, as the tongue 146 is moved up in the direction of the arrow 415 into engagement with a proximal notch 159 (FIGS. 4A, 4D). A syringe 174 is now attached to the port 172, with its plunger 174a pushed distally (in the direction of the arrow 416), so as to introduce fluid, air, liquid, gel, etc., here, for example, air through the tube 170 and the needle 166 into the balloon 134, inflating (expanding) it.

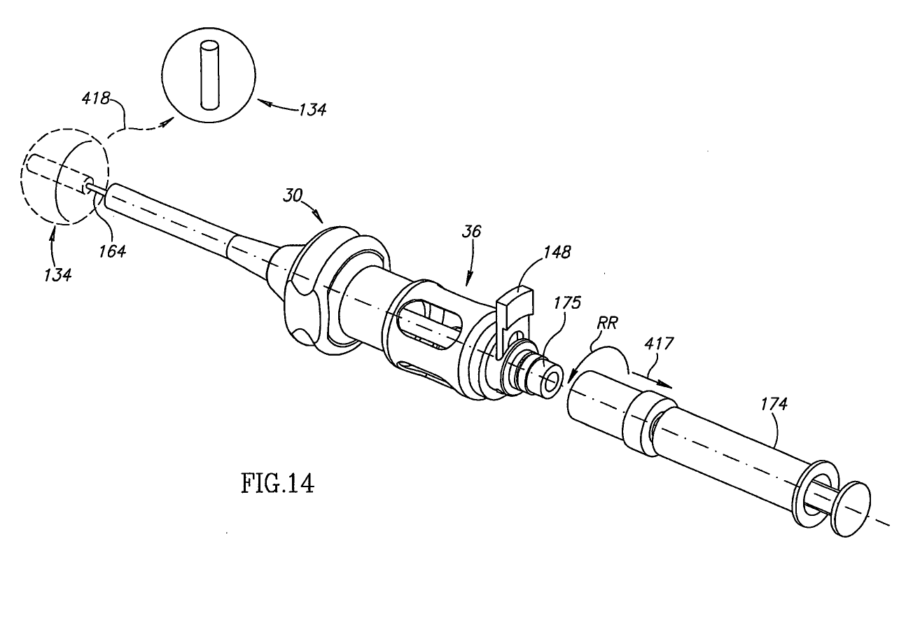

With the balloon 134 now inflated (expanded), the head 133 is separated (unscrewed) from the collar 175 of the shaft 162 (in the direction of the arrow RR). Once unscrewed, the head 133 is pulled proximally (away from the body), in the direction of arrow 417. This pulling moves the needle 166 proximately (as per arrow 417), retracting it into the tip 164. This retraction, coupled with resistance from the tip 164 (in particular the neck 196), releases the balloon 134 into the bladder 412 (as per the arrow 418), absent any forces from the body, as shown in FIG. 14.

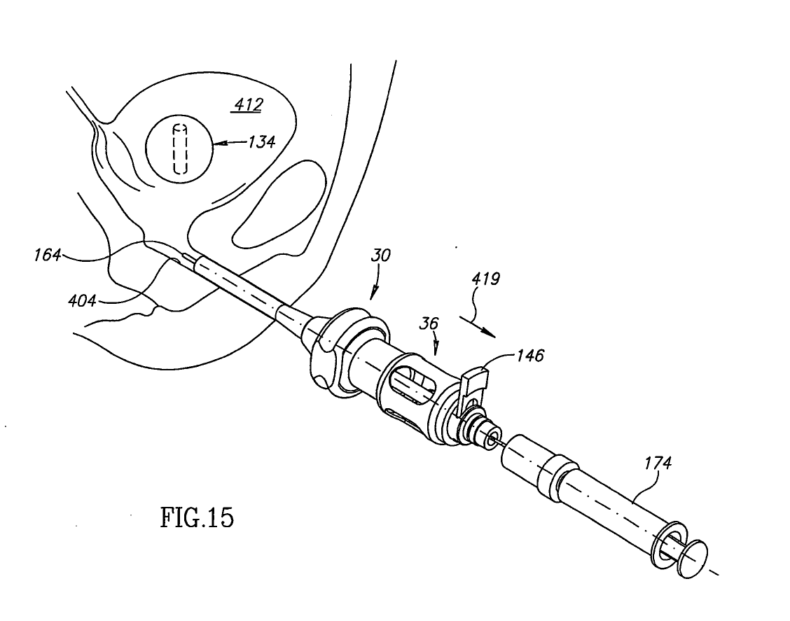

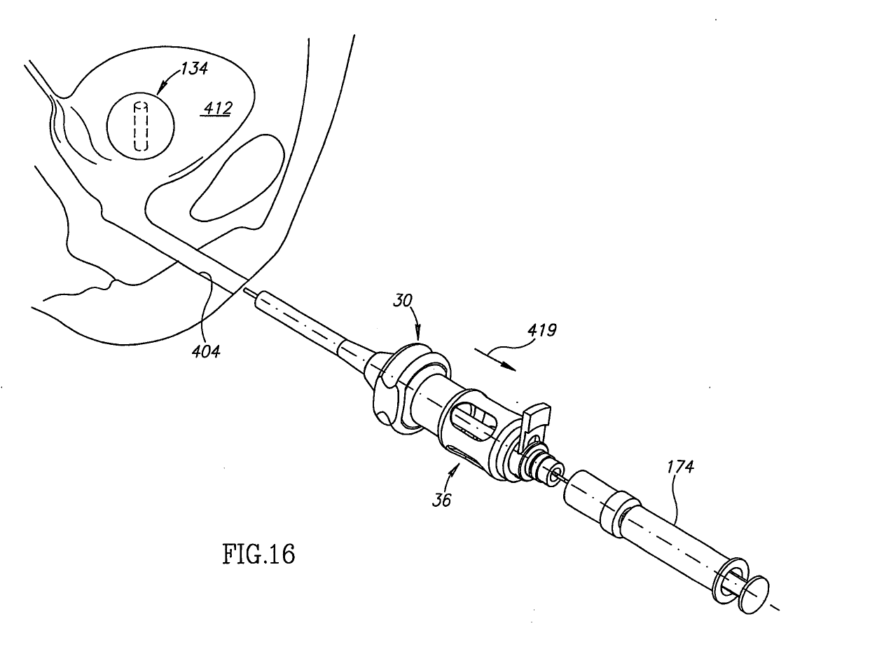

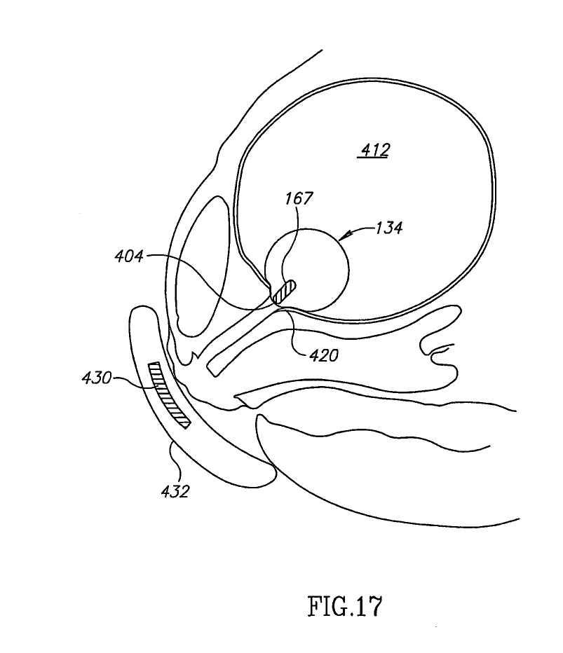

In FIG. 15, the magazine unit 36 and the urethral module 30, typically as a unit, are now pulled back (in the direction of the arrow 419), away from the urethra 404. Here, the balloon 134 floats in the bladder 412. Pulling continues in the direction of arrow 419 until the magazine unit 36 and urethral module 30 (as a unit) are completely removed from the body, as shown in FIG. 16.

In FIG. 17, the balloon 134 is maintained in the bladder neck 420 for example, by forces from a magnet 430 in a pad 432 in an undergarment or the like, as detailed in U.S. Pat. No. 6,293,923. This positioning of the balloon 134 in the bladder neck 420 blocks the flow of urine from the bladder 412, until the magnetic force is removed, whereby the balloon 134 will again floats free in the bladder 412, as also detailed and shown in U.S. Pat. No. 6,293,923.

When removal of the balloon 134 from the bladder 412 is desired, the urethral module 30 and mandrel 32 are assembled and inserted into the urethra 404, as detailed above and shown in FIGS. 6 and 7. The mandrel 32 is then removed, with the bladder located, as detailed above and shown in FIGS. 8 and 9.

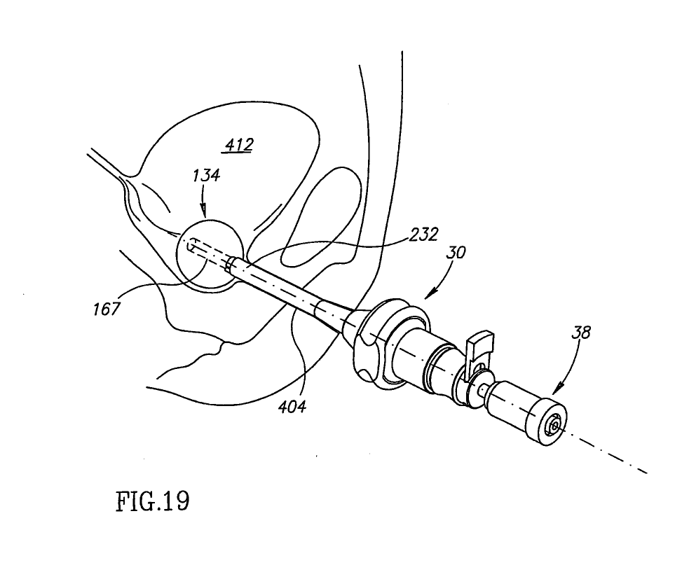

In FIG. 18, the retriever 38 is now inserted into the urethral module 30, in the direction of the arrow 468, until the tip 234, with the capturing member 232, has entered the bladder neck 420. In this position, the rod 224 of the retriever 38 is locked (tongue 268 engages notch 272). The magnet 232, attracts the balloon 134, so as to be in contact therewith, as shown in FIG. 19.

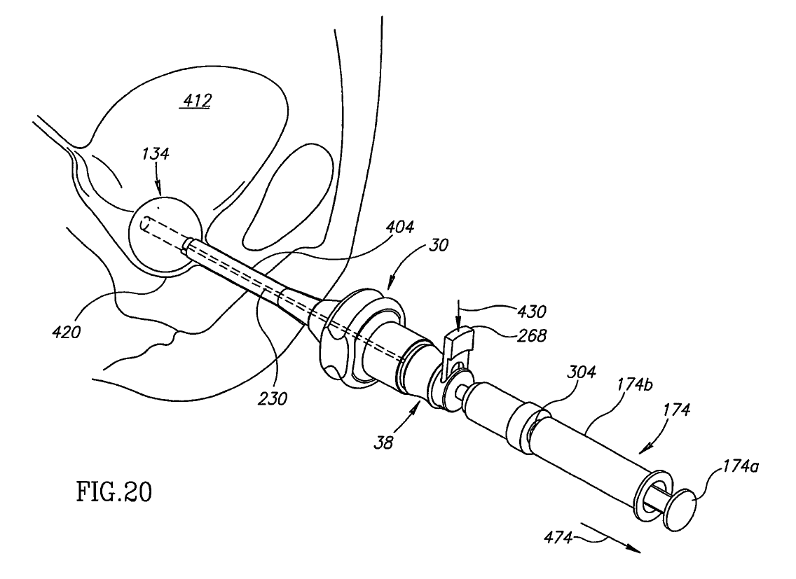

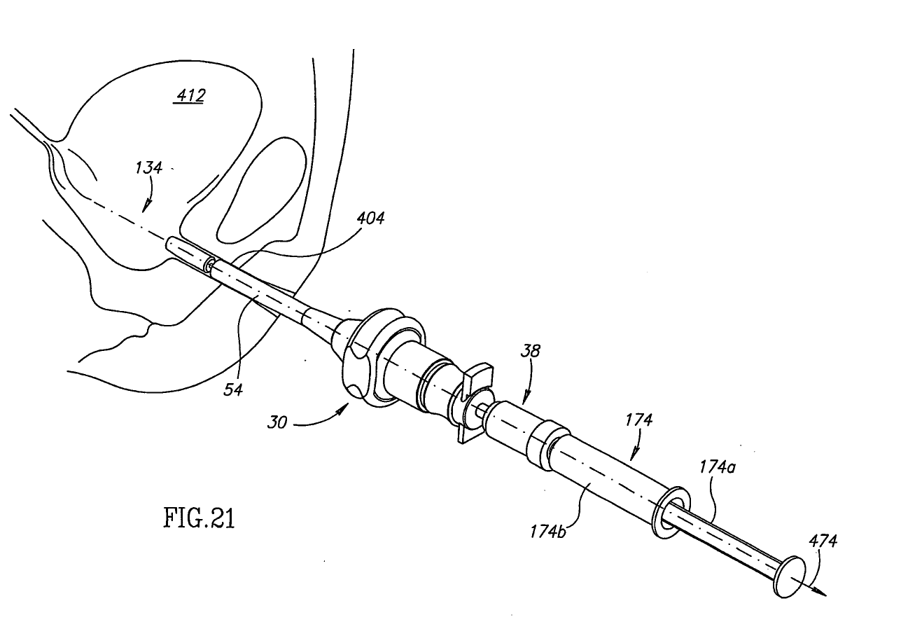

With the balloon 134 in contact with the magnet 232 of the tip 234, the locked rod 224 is released, as the tongue 268 disengages from the notch 272, as it is moved in the direction of the arrow 470. This unlocking allows the spring 252 to expand and move the rod 224 distally. Accordingly, the needle 230 moves distally, beyond the tip 234, such that the needle tip 230a punctures the balloon 134. Fluid (air, water, solution, gel, etc.) from the balloon 134 enters the needle 230, and coupled with suction from the syringe 174 (previously attached to the port 304), the fluid is pulled proximally (in the direction of the arrow 474, the direction the plunger 174a is moved) into the syringe 174, as shown in FIG. 20. Suction continues, as the plunger is pulled proximally (in the direction of the arrow 474) until the balloon 134 is deflated, as shown in FIG. 21.

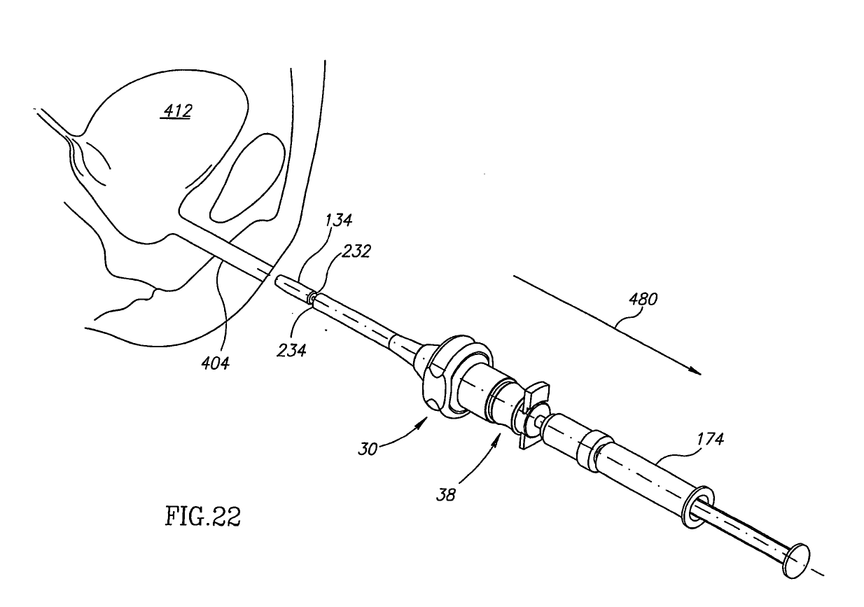

In FIG. 22, the retriever 38 with the now deflated balloon 134, on it, and the urethral module 30, as removed as a unit from the urinary tract by pulling in the direction of the arrow 480. Throughout this process the balloon 134 remains on the magnet 232 of the tip 234 due to the strong magnetic forces.

While the system 20 has been described above for a human female, it can be modified to accommodate the human male urinary system as well. Similarly, it can be modified for animals, both male and female, in accordance with the disclosure above.

While preferred embodiments of the present invention have been described, so as to enable one of skill in the art to practice the present invention, the preceding description, including instruments, components and combinations thereof, is intended to be exemplary only. It should not be used to limit the scope of the invention, which should be determined by reference to the following claims.

1. A magazine unit comprising:
a body including a chamber configured for receiving an implantable device, said body including a passageway extending therethrough;
a rod slidably received by said passageway in said body and of a length for sliding beyond said body, said rod including a channel for fluid passage extending at least substantially therethrough, and
a channel member in fluid communication with said channel, said channel member configured for receipt by said implantable device.

2. The magazine unit of claim 1, wherein said channel member includes a needle.

3. The magazine unit of claim 2, wherein said implantable device includes a fillable member.

4. The magazine unit of claim 3, wherein said fillable member is configured for temporarily receiving said needle.

5. The magazine unit of claim 4, wherein said fillable member includes a balloon.

6. The magazine unit of claim 5, wherein said balloon is substantially spherical in an expanded state.

7. The magazine unit of claim 1, additionally comprising a tube extending at least substantially along the length of said channel.

8. The magazine unit of claim 1, wherein said chamber is configured for receiving said implantable device in at least a partially inflated state.

9. The magazine unit of claim 7, wherein said tube is configured for receiving said channel member.

10. The magazine unit of claim 9, wherein said rod includes a head and a shaft, said head being separable from said shaft; and
said tube extends from at least said head into said shaft; and
said head is movable proximally and distally with respect to said body, such that movement of said head proximally pulls said tube and said channel member proximally, for releasing said implantable device from said channel member absent body forces.

11. An retrieving device comprising:
a body including a tubular segment extending therefrom;
a capturing member engaged within said tubular segment defining a tip portion of said tubular segment;
a needle including a tip and a bore extending therethrough, said needle coupled with a force generating member so as to be movable between a first position, where said needle tip has not moved beyond said tip portion, and a second position where said needle tip has moved beyond said tip portion, said force generating member configured for providing forces to said needle sufficient to pierce an implanatable device.

12. The retrieving device of claim 11, wherein said capturing member includes a magnet.

13. The retrieving device of claim 12, wherein said magnet includes a longitudinal groove along its outer surface for accommodating said needle.

14. The retrieving device of claim 11, additionally comprising a rod, said rod including a channel extending at least substantially therethrough, said channel in communication with the bore of said needle.

15. The retrieving device of claim 11, wherein said force generating member includes a spring.

16. A method for deploying an implantable member in a body organ comprising:
providing a magazine unit comprising:
a body including a chamber configured for receiving an implantable device, said body including a passageway extending therethrough;
a rod slidably received by said passageway in said body and of a length for sliding beyond said body, said rod including a channel for fluid passage extending at least substantially therethrough, and
a channel member in fluid communication with said channel, said channel member configured for receipt by said implantable member;
providing an implantable member in at least a partially expanded state onto said channel member; and
contracting said implantable member.

17. The method of claim 16, additionally comprising:
accessing the body organ by creating a tubular channel thereto; and
moving said implantable member through said tubular channel into said organ; and
expanding said implantable member.

18. The method of claim 17 additionally comprising:
moving at least a portion of said channel member into said rod for releasing said implantable member absent any body forces.

19. A method for retrieving an implantable member in at least one organ comprising:
accessing said organ;
capturing said implantable member;
piercing said implantable member with a device in fluid communication with a conduit; and
contracting said implantable member by suctioning the contents of said member through said conduit.

20. The method of claim 19, wherein said capturing is by magnetic forces.

21. The method of claim 19, wherein said piercing includes generating a force from a spring.

22. The method of claim 16 wherein said body organ is a urinary bladder.

23. The method of claim 19 wherein said organ is a urinary bladder.
